(19) United States
(12) Patent Application Publication
(10) Pub. No.: US 2011/0301046 A1
(43) Pub. Date: Dec. 8, 2011

(54) Methods of Determining Efficacy of Glucocorticoid Treatment of Eosinophilic Esophagitis

(75) Inventors: Marc E. Rothenberg, Cincinnati, OH (US); Julie Caldwell, Cincinnati, OH (US)

(73) Assignee: Children's Hospital Medical Center, Cincinnati, OH (US)

(21) Appl. No.: 13/132,295

(22) PCT Filed: Dec. 1, 2009
(86) PCT No.: PCT/US09/66282

§ 371 Date: Aug. 22, 2011

Related U.S. Application Data
(60) Provisional application No. 61/118,985, filed on Dec. 1, 2008.
(60) Provisional application No. 61/118,981, filed on Dec. 1, 2008.

(51) Int. Cl. C12Q 1/68; C40B 30/00; G01N 33/566

(52) U.S. Cl. 506/7; 435/6.12; 436/501

(57) ABSTRACT

The present invention concerns methods useful in diagnosing, identifying and monitoring the progression of eosinophilic esophagitis through measurements of gene products.

### CROSS REFERENCE TO RELATED APPLICATIONS

This application claims the benefit of priority under 35 U.S.C. §119(e) to U.S. Provisional Application Ser. No. 61/118,985, filed on Dec. 1, 2008, entitled METHODS OF DETERMINING EFFICACY OF GLUCOCORTICOID TREATMENT OF EOSINOPHILIC ESOPHAGITIS, and to U.S. Provisional Application Ser. No. 61/118,981, filed on Dec. 1, 2008, entitled IL-13 INDUCED GENE SIGNATURE FOR EOSINOPHILIC ESOPHAGITIS, each of which is incorporated herein by reference in its entirety.

### GOVERNMENT RIGHTS

This invention was made with U.S. Government support under NIH Research Project Grant program NIH 1 U19 AI070235-01. The U.S. Government may have certain rights in the subject matter hereof.

### BACKGROUND

All publications herein are incorporated by reference to the same extent as if each individual publication or patent application was specifically and individually indicated to be incorporated by reference. The following description includes information that can be useful in understanding the present invention. It is not an admission that any of the information provided herein is prior art or relevant to the presently claimed invention, or that any publication specifically or implicitly referenced is prior art.

Eosinophilic esophagitis (EE) is characterized by an abnormal accumulation of eosinophils in the esophageal mucosa and is often linked to an allergic etiology. Symptoms include nausea, vomiting, abdominal pain, chest pain, heartburn, regurgitation, dysphagia, food impaction, poor appetite, early satiety, fussiness, and poor weight gain. The current treatment of EE is with swallowed glucocorticoids such as fluticasone propionate (FP) and beclothemasone, man-made steroids that are related to naturally occurring steroid hormone, cortisol or hydrocortisone, produced by the adrenal glands. However, only a subset of EE patients experience remission from the disease following treatment with topical FP.

Thus, there is a need in the art for a better understanding of the molecular mechanisms of both EE and remission induced by FP, as well as methods of treatment.

### SUMMARY

Embodiments of the invention relate to a method of determining the prognosis of eosinophilic esophagitis (EE) after glucocorticoid treatment in an individual and includes: determining the presence or absence of elevated FKBP51 expression; and prognosing a responsive case of eosinophilic esophagitis after glucocorticoid treatment based upon the presence of elevated FKBP51 expression. In some embodiments, the presence of, for example, a glucocorticoid-responsive gene expression profile, and the like, is indicative of a nonaggressive form of EE.

In other embodiments, a method for determining if an EE patient has been exposed to a steriod drug, includes analysis of a gene expression profile of EE-associated genes, wherein the analysis includes, for example, any or all of the genes in FIG. 1. In other embodiments, the analysis includes detection of a presence or an absence of elevated FKBP51 expression and the like. In some embodiments, the method identifies, for example, patient compliance with taking a medicine and the like. In other embodiments, the method determines the efficacy, for example, of a steroid drug and the like.

In some embodiments, a method of diagnosing an EE subtype includes: determining the presence or absence of at least one glucocorticoid-responsive transcript; and diagnosing the EE subtype based upon the presence or absence of the transcript or transcripts. In other embodiments, the EE subtype is responsive, for example, to fluticasone propionate (FP) treatment and the like. In alternative embodiments, the glucocorticoid-responsive transcript includes the expression of a gene described in FIG. 1(B) herein and the like. In some embodiments, the glucocorticoid is, for example, FP and the like.

In other embodiments, a method of treating EE in an individual includes: determining the presence, for example, of a glucocorticoid-responsive gene expression profile and the like; and treating the individual based upon the profile. In some embodiments, the glucocorticoid-responsive gene expression profile includes, for example, the expression of FKBP51 and the like.

In some embodiments, FKBP51 is expressed in, for example, peripheral blood mononuclear cells and/or esophageal epithelial cells and the like.

In other embodiments, a device for diagnosing an EE subtype, includes: a gene array, for example, integrated with the expression profile of, for example, one or more glucocorticoid-responsive transcripts.

In some embodiments there is provided a kit for the detection of expression levels of one or more genes associated with EE, wherein the kit can include: complementary oligonucleotide probes, for example, and the like, to subsequences of one or more genes. In some embodiments, the kit can include probes, wherein the probes can be used in one or more of, for example, a gene chip, a PCR protocol and the like.

Other embodiments provide a method of prognosis includes the steps of:

a. in an isolated sample, previously contacted with, for example, a steroid drug, measuring, for example, the expression level of FKBP51;

b. comparing the expression level measured in (a) to a pre-determined value, thereby obtaining a prognosis.

In some embodiments, a method for determining exposure to a steroid drug, for example, includes the steps of:

a. in an isolated sample measuring the expression level, for example, of any or all of the genes in FIG. 1; and

b. comparing the expression level measured in (a) to a pre-determined value, thereby obtaining a determination of exposure to a steroid drug. In other embodiments, the sample is isolated after being contacted with, for example, fluticasone propionate and the like.

### BRIEF DESCRIPTION OF THE FIGURES

FIG. 1 depicts the identification of glucocorticoid-regulated genes in EE patients that respond to FP treatment. (A) Analysis of microarray data to identify glucocorticoid-regulated transcripts. RNA from distinct patient populations was subjected to chip analysis using Affymetrix Human Genome U 133 Plus 2.0 GeneChips®. The groups are composed of the following numbers of patients: normal (NL), n=14; EE, n=14; EE, FP responders (EE R), n=13; EE, FP non-responders, n=8. Using the average expression for each patient group, the subset of genes differentially regulated between normal (NL) and EE patients was identified by t-test (p<0.01). The subset of genes differentially regulated between normal patients and EE patients that respond to FP (EE R) was then identified by 2-fold change filter. Subsequently, the subset of genes differentially regulated between NL and EE patients was subtracted from those differentially regulated between NL and EE R. These transcripts were then subjected to ANOVA, p<0.01. (B) Genes identified by the analysis described in (A). Average expression levels of the transcripts identified as being glucocorticoid-regulated are shown for the normal, EE, and EE FP responder patient populations. Upregulated genes are represented by red, and downregulated genes are colored blue. The magnitude of change in expression is proportional to the darkness of the color. (C) Genes exhibiting increased transcript levels in responder patients. Average gene expression levels are indicated for normal, EE, EE FP responder, and EE FP non-responder patients. (D) Genes showing decreased transcript levels in FP responder patients. Average gene expression levels are indicated for normal, EE, EE FP responder, and EE FP non-responder patients.

FIG. 2 depicts the verification of transcript levels of glucocorticoid-regulated genes by real-time PCR analysis. (A) Microarray data for three glucocorticoid-regulated genes. The average expression level for each patient group (normal, EE, Flovent® (fluticasone propionate) responders, Flovent® non-responders) is expressed as fold-change compared to the normal patients. (B) Real-time PCR analysis for three glucocorticoid-regulated genes. The transcript levels for the indicated gene for each patient population was quantified by real-time PCR and normalized to glyceraldehyde-3-phosphate dehydrogenase (GAPDH) levels. The graph displays the average fold-change of each transcript in a particular patient population compared to that of the normal controls. *, p<0.05; **, p<0.01; ***, p<0.001.

FIG. 3 depicts FKBP51 induction following glucocorticoid treatment. (A) Peripheral blood mononuclear cells (PBMCs) were isolated from a patient blood sample and cultured ex vivo. The cells were treated with the indicated concentration of FP for 24 h. RNA was isolated, cDNA synthesis was performed, and real-time PCR analysis was conducted. FKBP51 transcript levels were normalized to GAPDH levels. (B) Primary esophageal epithelial cells were cultured from the biopsy of an EE patient with active disease. Cells were treated or not with 10⁻⁶ M FP for 24 h. Protein extracts were prepared and subjected to SDS-PAGE and western blot analysis for FKBP51 and actin (left). FKBP51 and actin levels were quantified by densitometry analysis. The ratio of FKBP51 to actin is shown (right). (C) TE-7 cells were treated with the indicated dose of FP for 24 h. Protein extracts were then prepared and subjected to SDS-PAGE and western blot analysis for FKBP51 and actin (top). Densitometry analysis was performed and the fold-change in the ratio of FKBP51 to actin expression is graphed (bottom). (D) TE-7 cells were treated for the indicated amount of time with 10⁻⁷ M FP. RNA was isolated, cDNA synthesis was performed, and real-time PCR analysis to detect FKBP51 and GAPDH transcripts was done (left). In a separate experiment, protein extracts were prepared and then subjected to SDS-PAGE and western blot analysis for FKBP51 and actin (right, top). Following densitometry analysis, the fold-change in the ratio of FKBP51 to actin signal was graphed (right, bottom)*, p<0.05; **, p<0.01.

FIG. 4 depicts IL-13 induced transcript levels of two representative genes are reversed by FP treatment. TE-7 cells were treated as indicated with IL-13 (100 ng/ml) and/or the indicated dose of FP for 24 h. RNA was isolated and cDNA synthesis was performed. Transcript levels of eotaxin-3 (A) and serpinb4 (B) were determined by real-time PCR analysis and normalized to GAPDH levels. *, p<0.05; **, p<0.01.

FIG. 5 depicts increased baseline FKBP51 levels impact glucocorticoid repression of IL-13-induced eotaxin-3 promoter activity. (A) Schematics of constructs used in this study. GRE, glucocorticoid response element. CMV, cytomegalovirus promoter. (B) Cells were transfected with Phrl-TK, pEotaxin-3, and Pcdna3.1 or Pfkbp51. Cells were then treated with IL-13 (10 ng/ml) and/or FP (10⁻⁶ M) for 24 h. Protein lysates were then collected, and firefly and Renilla luciferase activity for each sample were quantified by dual luciferase assay (Promega). Eotaxin-3 promoter activity is expressed as the ratio of firefly to Renilla luciferase activity. **, p<0.01; ***, p<0.001.

FIG. 6 depicts FP reduces IL-13-induced eotaxin-3 promoter activity, but does not impact the contribution of the eotaxin-3 3′ UTR to transcript stability. Cells were transfected with Phrl-TK and pEotaxin-3 (A) or pEotaxin-3 3′UTR (B) for 48 h. Cells were then treated with IL-13 (100 ng/ml) and/or FP (10⁻⁶ M) for 24 h. Protein lysates were then collected, and firefly and Renilla luciferase activity for each sample was quantified by dual luciferase assay (Promega). Eotaxin-3 promoter activity is expressed as the ratio of firefly to Renilla luciferase activity. **, p<0.01.

### DESCRIPTION

### Definitions

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russell, Molecular Cloning: A Laboratory Manual 3^{rd} ed., Cold Spring Harbor Laboratory Press (Cold Spring Harbor, N.Y. 2001), provide one skilled in the art with a general guide to many of the terms used in the present application.

As used herein, the term “level” includes a gage of, or measure of the amount of, or concentration of a transcription product, for instance mRNA, or a translation product, for instance a protein or polypeptide. A level of RNA expression can be expressed in units such as transcripts per cell or nanograms per microgram of tissue. A level of a polypeptide can also be expressed as nanograms per microgram of tissue or nanograms per milliliter of a culture medium, for example. Alternatively, relative units can be employed to describe an expression level. For example, when an assay has an internal control, for instance a control gene, for example glyceraldehyde 3-phosphate dehydrogenase (GAPDH), for which the expression level is either known or can be accurately determined, unknown expression levels of other genes can be compared to the known internal control.

Once an expression level is determined for a gene, a profile can be created. As used herein, the term “profile,” for example a gene expression profile, refers to a repository of the expression level data that can be used to compare the expression levels of different genes, in whatever units are chosen. The term “profile” is also intended to encompass manipulations of the expression level data derived from a cell, tissue or individual. For example, once relative expression levels are determined for a given set of genes, the relative expression levels for that cell, tissue or individual can be compared to a standard to determine if expression levels are higher or lower relative to the same genes in a standard. Standards can include any data deemed by one of skill to be relevant for comparison. A standard can be prepared by determining the average expression level of a gene in a normal population, a normal population being defined as subjects that do not have EE. A standard can also be prepared by determining the average expression level of a gene in a population of individuals with EE.

As used herein, the term “determining,” and grammatical derivatives thereof, such as, but not limited to “determine,” or “determined,” can include measuring the expression level, for example, the amount or concentration of a nucleic acid or protein marker of the invention. The term thus can refer to use of materials, compositions and methods of the present invention for qualitative and quantitative assessment. A qualitative determination of the level of a marker can include comparing the level of a marker in a sample with the level of the marker in a control sample or with the level of the marker obtained from the same patient but at a different time point. A quantitative determination includes measuring the amount or concentration of the level of a nucleic acid or protein that is encoded by or that corresponds to the particular marker. For example, detecting a change in expression levels can include quantifying a change of any value between 10% and 90%, or of any value between 20% and 80%, 30% and 70%, 40% and 60% or over 100%, of a marker of the invention relative to a control. Detecting an increase in gene expression levels can include quantifying a change of any value between 1.5 fold to 10000 fold or more of any of the markers of the invention relative to a control. An increase in gene expression levels can include changes of 2, 5, 10, 25, 50, 100, 1000 fold or more.

As used herein, the term “detect” and all other forms of the root word “detect” can refer to the ascertainment of the presence or absence of one or more markers, quantization of one or more targets, or assessment of the presence or absence of a threshold value of one or more markers. A threshold value can be determined experimentally, empirically, or theoretically. A threshold value can also be arbitrarily selected based upon the existing experimental and/or clinical conditions, as would be recognized by a person of ordinary skill in the art.

As used herein, the term “gene chip” refers to a matrix, the basic material of which is, for example, glass or nylon, onto which DNA fragments are immobilized, it being possible for the application of the DNA to be carried out for example by (a) a photolithographic process (DNA is synthesized directly on the array matrix), (b) a microspotting process (externally synthesized oligonucleotides or PCR products are applied to the matrix and covalently bonded thereto), or (c) by a microspraying process (externally synthesized oligonucleotides or PCR products are sprayed onto the matrix without contacting by an ink-jet printer) (cf. R. Rauhut, Bioinformatik (Bioinformatics), pp 197-199, ed: Wiley-VCH Verlag GmbH, Weinheim, 2001). A gene chip that represents genomic sequences of an organism is typically referred to as a genomic DNA gene chip. The analysis of the measured values obtained with the aid of a gene chip is gene chip analysis.

As used herein the term “microarray” refers to a set of oligonucleotide probes arranged on a solid matrix, such as a microscope slide or silicon wafer. The oligonucleotide probes are generally 10-50 nucleotides in length, preferably 15-40 nucleotides, more preferably 20-30 nucleotides, most preferably about 24 nucleotides. Each probe has a defined locus. RNA derived from a sample obtained from the patient can be labeled and hybridized to the oligonucleotide probes on the microarray to detect the level of gene expression.

As used herein, the term “transcript” can refer to an RNA molecule that is derived through the process of transcription from DNA. Transcripts can also be represented in some situations by proteins translated from RNA transcripts. A “glucocorticoid” is a steroid hormone capable of binding to the glucocorticoid receptor. A “glucocorticoid-responsive transcript” refers to an RNA molecule or molecules whose expression is either increased or decreased, by 1.5, 2, 5, 10, 25, 50, 100, 1000, 10000 fold or more, in the presence of a glucocorticoid.

As used herein, the term “subsequence” refers to any part of a polynucleotide sequence that is less than the entire polynucleotide sequence, and that would be suitable to perform the method of analysis. A person skilled in the art can choose the position and length of a subsequence by applying routine experiments. For example, a subsequence of a polynucleotide can be any contiguous sequence of at least about 10, about 25, about 50, about 100, about 200, about 300, about 400, about 800, or about 1,000 nucleotides, or more.

As used herein, the term “treating” or “treatment,” with respect to disease encompasses (1) preventing the disease, for example, causing the clinical symptoms of the disease not to develop in an animal that is exposed to or predisposed to the disease but does not yet experience or display symptoms of the disease, (2) inhibiting the disease, for example, arresting the development of the disease or its clinical symptoms, or (3) relieving the disease, completely or partially, for example, causing regression of the disease or its clinical symptoms. It will be appreciated by those skilled in the art that treatment extends to prophylaxis as well as the treatment of inflammation or other symptoms.

As used herein, the term “presence” refers to when a molecule can be detected using a particular detection methodology. Also as used herein, the term “absence” refers to when a molecule cannot by detected using a particular detection methodology.

As used herein, the term “elevated” encompasses activity that is increased above the level found typically in cells or tissue from an individual free of EE relative to the same type of cell or tissue from an individual diagnosed with EE. Generally, elevated activity is at least about 1.5, 2, 5, 10, 25, 50, 100, 1000, 10000 fold, or more greater than that in corresponding cells or tissues from an individual free of EE.

As used herein, the term “administering” and grammatical derivatives thereof, refers to, the contacting of a compound, reagent, or material directly to a cell or tissue or to the environment that surrounds and/or is adjacent to the cell or tissue. The term “administer” also can refer to any route of introducing or delivering to an individual a compound, reagent, or material to perform its intended function. Administration in most cases can be carried out by any suitable route, including, but not limited to, topical, transdermal, intranasal, vaginal, rectal, oral, subcutaneous intravenous, intra-arterial, intramuscular, intraosseous, intraperitoneal, epidural and intrathecal.

As used herein, the term “patient” encompasses includes an individual with symptoms of and or suspected of having EE. Patient includes human beings, but can also include animals generally. Patients generally can be female or male and person(s) of all ages.

As used herein, the term “compliance,” when used in the context of a prescribed dosing regimen, can be, in many embodiments, a neutral term that denotes not only the degree of conformance to the dosing regimen but can also denote the degree of deviation from the dosing regimen.

As used herein, the term “non-responsive,” in relation to EE means patients who have not had a reasonable clinical response, for example, a 5%, 10% 15%, 25%, 35%, 45%, 55%, 65%, 75%, 85% or more reduction in a clinically recognized indicia of responsiveness from a patient's baseline score after treatment with one or more clinical courses of anti-EE medication (for example, steroid drugs).

The term “oligonucleotide” refers to a relatively short polynucleotide, typically less than or equal to 150 nucleotides long, for example, between 5 and 150 nucleotides in length, preferably between 10 and 100 nucleotides in length, or more preferably between 15 and 50 nucleotides in length. As used herein, the term “oligonucleotide” can encompass longer or shorter polynucleotide chains. An “oligonucleotide” can hybridize to other polynucleotides or target nucleic acids, therefore serving as a probe for polynucleotide detection. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

As used herein, the term “complementary” refers to the concept of sequence complementarity between regions of two polynucleotide strands. It is known that an adenine base of a first polynucleotide region is capable of forming specific hydrogen bonds (“base pairing”) with a base of a second polynucleotide region that is antiparallel to the first region if the base is thymine or uracil. Similarly, it is known that a cytosine base of a first polynucleotide strand is capable of base pairing with a base of a second polynucleotide strand that is antiparallel to the first strand if the base is guanine. A first region of a polynucleotide is complementary to a second region a different polynucleotide if, when the two regions are arranged in an antiparallel fashion, at least one nucleotide of the first region is capable of base pairing with a base of the second region. Therefore, it is not required for two complementary polynucleotides to base pair at every nucleotide position. “Complementary” can refer to a first polynucleotide that is 100% or “fully” complementary to a second polynucleotide and thus forms a base pair at every nucleotide position. “Complementary” also can refer to a first polynucleotide that is not 100% complementary (e.g., 90%, 80%, 70% complementary or less) contains mismatched nucleotides at one or more nucleotide positions.

As used herein, the term “probe” encompasses a polymer (e.g. a DNA, RNA, PNA, LNA chimera, linked polymer as well as combinations thereof (for example, an LNA/DNA chimera)) designed to hybridize sequence-specifically to a target sequence of interest. An “oligonucleotide probe” refers to a nucleic acid probe, of either DNA or RNA, used to detect the presence of a complementary target sequence by hybridization with the target sequence. In some cases, a probe can be designed to hybridize partially with a target sequence of interest.

As used herein, the term “prognosis” means a prediction of the probable outcome and/or course of a disease, it can be measured by reference to any suitable parameter recognized by those of skill in the art.

As used herein, the term “sample” refers to a biological material that is isolated from its natural environment and contains a polynucleotide. A “sample” according to the invention can include a purified or isolated polynucleotide, or it can include a biological sample such as a tissue sample, a biological fluid sample, or a cell sample including a polynucleotide. A biological fluid can be, for example, blood, plasma, sputum, urine, cerebrospinal fluid, lavages, biopsy, for example esophageal biopsy or esophageal mucosal biopsy, and leukophoresis samples. Useful samples of the present invention can be obtained from different sources, including, for example, but not limited to, from different individuals, different developmental stages of the same or different individuals, different diseased individuals, normal individuals, different disease stages of the same or different individuals, individuals subjected to different disease treatments, individuals subjected to different environmental factors, individuals with predisposition to a pathology, individuals with exposure to an infectious disease. Useful samples can also be obtained from in vitro cultured tissues, cells, or other polynucleotide containing sources. The cultured samples can be taken from sources including, but are not limited to, cultures (for example, tissue or cells) cultured in different media and conditions (for example, Ph, pressure, or temperature), cultures (for example, tissue or cells) cultured for different period of length, cultures (for example, tissue or cells) treated with different factors or reagents (for example, a drug candidate, or a modulator), or cultures of different types of tissue or cells.

One skilled in the art will recognize many methods and materials similar or equivalent to those described herein that can be used in the practice of the present subject matter. Indeed, the present subject matter is in no way limited to the methods and materials described.

“EE” as used herein is an abbreviation for Eosinophilic Esophagitis.

“ER” as used herein is an abbreviation for Estrogen receptor.

“FP” as used herein is an abbreviation for Fluticasone propionate. FP is a white to off-white powder, with the empirical formula C₂₅H₃₁F₃O₅S and a molecular weight of 550.6. Fluticasone refers to the synthetic, trifluorinated, corticosteroid having the chemical name of S-fluoromethyl-6α,9-difluoro-11β-hydroxy-16α-methyl-3-oxoandrosta-1,4-diene-17β-carbothioate, 17-propionate, and salts and derivates thereof. Only a subset of EE patients experience remission from the disease following treatment with topical FP.

As used herein, the term “FKBP51” is an abbreviation of FK506-binding protein 5. FKBP51 is a 51 Kd member of the FK506 Binding Protein (FKBP) of immunophilins. This family of proteins is defined by the ability to bind the immunosuppressive drug FK506.

As described herein, FP-regulated transcripts not normally expressed in EE patients were identified and correlated with FP responsiveness. To understand the molecular mechanisms of EE and remission induced by FP, transcript levels of biopsy samples from distinct patient populations were compared globally by whole genome microarray analysis. Thirty-two glucocorticoid-responsive transcripts exhibited altered levels in normal patients compared to EE patients that responded to FP treatment. Among these, FK506 binding protein 5 (FKBP51) was identified. Microarray analysis demonstrated a 4-fold increase in FKBP51 transcripts in FP-responders compared to control and active EE individuals. FKBP51 transcript levels were increased in peripheral blood mononuclear cells and esophageal epithelial cells (primary cultures and the TE-7 cell line) following FP treatment, and FKBP51 protein levels were increased following FP treatment of primary esophageal epithelial cells and the esophageal epithelial cell line. FKBP51 overexpression in TE-7 cells reduced FP-mediated inhibition of IL-13-induced eotaxin-3 expression by 50%.

Some embodiments of the invention relate to a method of diagnosing EE in a human, the method comprising the steps of a) obtaining a biological test sample from a human to be diagnosed, preferably a human suspected of having or being predisposed to EE, b) analyzing the transcriptome of the sample from the patient suspected of having or being predisposed to EE, c) comparing the transcriptome from the patient suspected of having or being predisposed to BE to a sample from a healthy individual or population, d) identifying a difference in the expression pattern in the transcriptome of the patient suspected of having or being predisposed to EE from a healthy individual or population, and d) diagnosing EE in the patient to be diagnosed based on the differences between the two samples, wherein the presence of differences in the transcriptome between the test sample as compared to the control sample is indicative of the risk and/or presence of EE.

As further described herein, the results demonstrate that swallowed glucocorticoid treatment directly impacts esophageal gene expression of EE patients. In particular, elevated FKBP51 transcript levels identify glucocorticoid exposure in vivo and distinguish FP-responder patients from untreated active EE patients. FKBP51 reduces glucocorticoid-mediated inhibition of IL-13 signaling in epithelial cells, suggesting FKBP51 can influence patient FP responsiveness. Accordingly, esophageal FKBP51 levels have diagnostic and prognostic significance in EE.

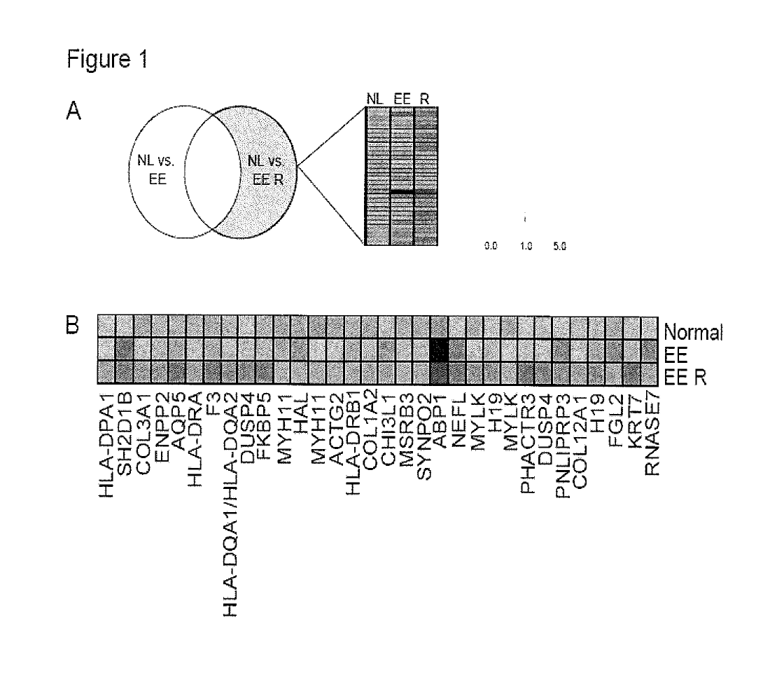

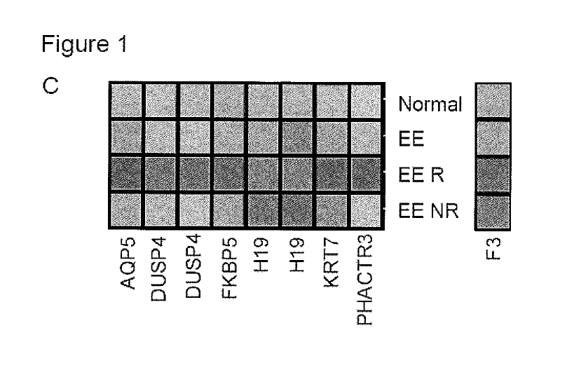

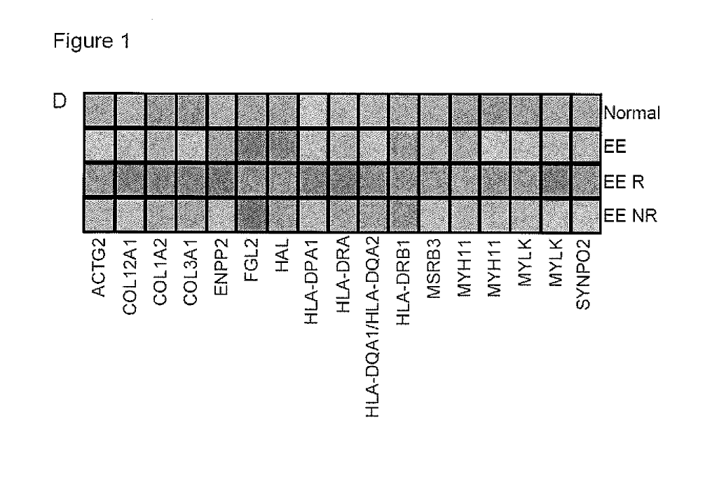

In one embodiment, the present invention provides a method of diagnosing an EE subtype by determining the presence or absence of one or more glucocorticoid-responsive transcripts, where the presence of one or more glucocorticoid-responsive transcripts is indicative of the EE subtype. In another embodiment, the EE subtype is responsive to FP treatment. In another embodiment, the glucocorticoid-responsive transcript includes the expression of a gene described in FIG. 1(B) herein. In another embodiment, the glucocorticoid is fluticasone propionate.

In one embodiment, the present invention provides a method for determining the prognosis of EE in an individual by determining the presence or absence of a glucocorticoid-responsive gene expression profile, where the presence of the glucocorticoid-responsive gene expression profile is indicative of a nonaggressive form of EE.

In one embodiment, the present invention provides a method of treating EE in an individual by determining the presence of a glucocorticoid-responsive gene expression profile, and treating the individual. In another embodiment, the glucocorticoid-responsive gene expression profile includes the expression of FKBP51. In another embodiment, FKBP51 is expressed in peripheral blood mononuclear cells and/or esophageal epithelial cells.

The present invention is also directed to a kit for the detection of expression levels of one or more genes, and can include an array of immobilized oligonucleotide probes complementary to subsequences of said one or more genes. Likewise, the kit can include materials for detection of genes; gene expression; expression, accumulation, and/or localization of proteins; and the like, including, for example, reagents, equipment, and/or instrumentation for ELISA, gene-chip expression analysis, RT-PCR, and the like. The kit is an assemblage of materials or components, including at least one of the inventive compositions. Thus, in some embodiments the kit contains a composition including polynucleotides encoding glucocorticoid-responsive transcripts, as described above.

The exact nature of the components configured in the inventive kit depends on its intended purpose. For example, some embodiments are configured for the purpose of detecting an expression profile of glucocorticoid-regulated genes.

Instructions for use can be included in the kit. “Instructions for use” typically include a tangible expression describing the technique to be employed in using the components of the kit to effect a desired outcome, such as to prepare a gene array for the diagnosis and/or prognosis of efficacy of glucocorticoid treatment of eosinophilic esophagitis. Optionally, the kit also contains other useful components, such as, diluents, buffers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in preparing a nanoconjugate. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing a solution of polynucleotides encoding the FKBP51 transcript. The packaging material generally has an external label which indicates the contents and/or purpose of the kit and/or its components.

One skilled in the art will recognize many methods and materials similar or equivalent to those described herein, which can be used in the practice of the present invention. Indeed, the present invention is in no way limited to the methods and materials described.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1

### General

Eosinophilic esophagitis (EE) is characterized by an abnormal accumulation of eosinophils in the esophageal mucosa. A subset of EE patients experience remission from the disease following treatment with topical fluticasone propionate (FP). FP-regulated transcripts not normally expressed in EE patients were identified and correlated with FP responsiveness. To understand the molecular mechanisms of EE and remission induced by FP, transcript levels of biopsy samples from distinct patient populations were compared globally by whole genome microarray analysis. Thirty-two glucocorticoid-responsive transcripts exhibited altered levels in normal patients compared to EE patients that responded to FP treatment. Among these, FK506 —binding protein 5 (FKBP51) was identified. Microarray analysis demonstrated a 4-fold increase in FKBP51 transcripts in FP-responders compared to control and active EE individuals. FKBP51 transcript levels were increased in peripheral blood mononuclear cells and esophageal epithelial cells (primary cultures and the TE-7 cell line) following FP treatment, and FKBP51 protein levels were increased following FP treatment of primary esophageal epithelial cells and the esophageal epithelial cell line. FKBP51 overexpression in TE-7 cells reduced FP-mediated inhibition of IL-13-induced eotaxin-3 expression by 50%. The results demonstrate that swallowed glucocorticoid treatment directly impacts esophageal gene expression of EE patients. In particular, elevated FKBP51 transcript levels identify glucocorticoid exposure in viva and distinguish FP-responder patients from untreated active EE patients. FKBP51 reduces glucocorticoid-mediated inhibition of IL-13 signaling in epithelial cells, suggesting that FKBP51 can influence patient FP responsiveness. Esophageal FKBP51 levels have diagnostic and prognostic significance in EE.

### Example 2

### Identification of Glucocorticoid-Regulated Genes in the Esophagus

A subset of EE patients that are treated with swallowed fluticasone propionate (FP) respond to this treatment by showing a decrease in esophageal eosinophils, epithelial hyperplasia, and symptom improvement. In order to understand the molecular mechanism accounting for the differential response of EE patients to this drug, genome-wide expression analysis was performed, involving esophageal biopsy samples derived from four distinct patient populations including normal patients, FE patients, EE patients that responded to FP treatment, and EE patients that failed to respond to FP treatment. To identify FP-regulated genes in the esophagus, the research aimed to identify the subset of genes that were regulated by FP treatment but not normally expressed in the esophagus. Accordingly, genes exhibiting differential expression between normal and EE patients were subtracted from the subset of genes that were differentially expressed between normal patients and FP responder patients (FIG. 1A). From this analysis, 32 transcripts were identified that were differentially expressed in FP responder patients compared to normal patients but were not included in the genes differentially expressed in EE patients at baseline (FIG. 1B). The expression profiles for these particular genes in all four patient groups were compared. Of the subset with increased levels in responder patients, one gene (F3) was identified that exhibited increased expression in both responder and non-responder patients (FIG. 1C). The remaining 8 transcripts representing 6 genes exhibited increased expression in responder but not non-responder patients. Of the 17 transcripts that represented 15 genes with decreased levels in responder patients, all showed levels in non-responder patients that were similar to normal patients (FIG. 1D). Notably, 4 MHC class II genes were identified, as well as 3 genes of the collagen family.

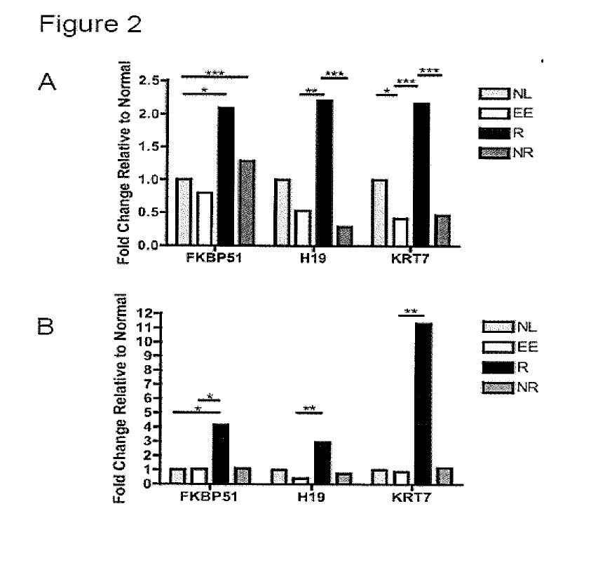

The expression pattern of three of the identified candidate genes was validated by real-time PCR analysis. FKBP51, KRT7, and H19 showed increased transcript levels in FP responder patients compared to untreated patients by both microarray (FIG. 2A) and real-time PCR analysis (FIG. 2B). Transcripts of these candidate genes exhibited decreased levels in FP non-responders compared to FP responder patients according to both analyses (FIG. 2A, 2B).

### Example 3

### FKBP51 Expression Patterns Following FP Treatment of PBMCs and Epithelial Cells

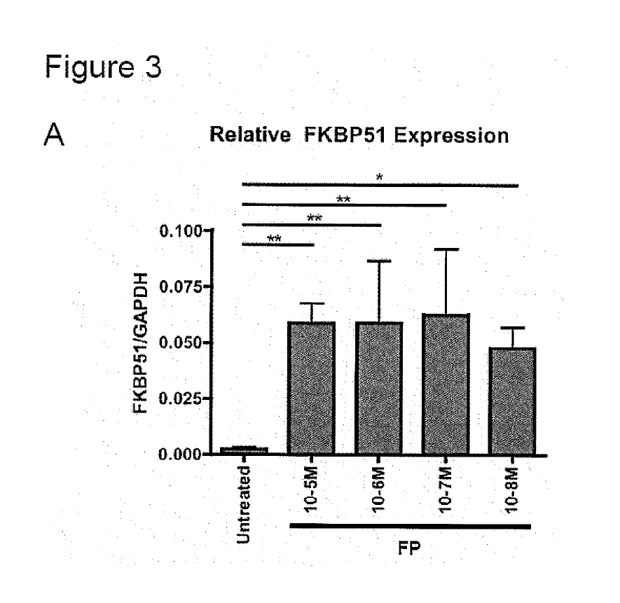

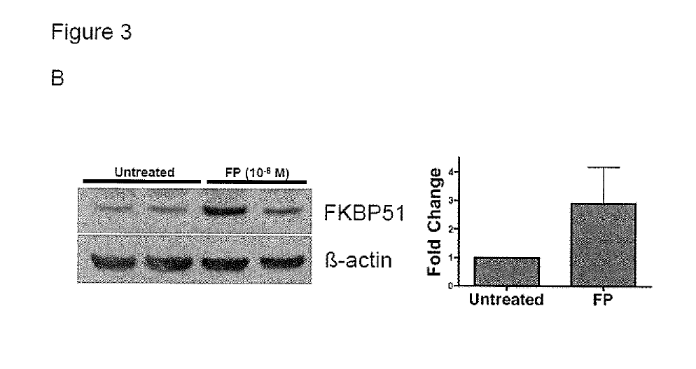

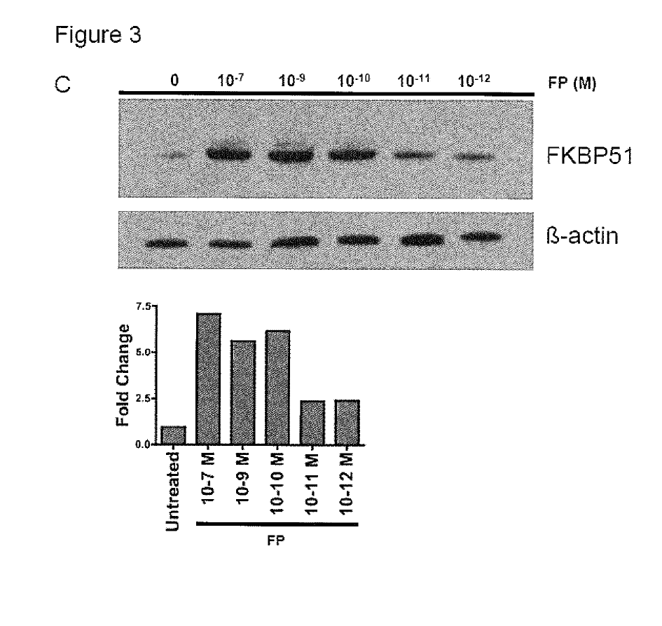

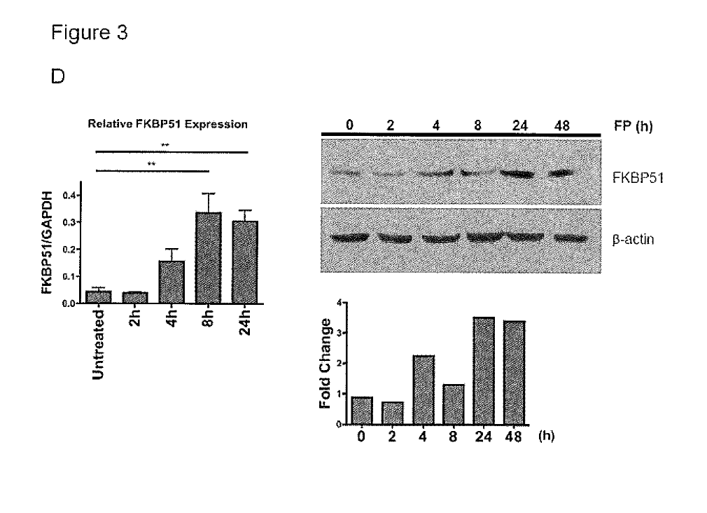

One of the glucocorticoid-regulated genes identified, FKBP51, exhibited increased expression in FP responder patients compared to normal and EE patients. On average, FKBP51 levels were 4-fold higher in FP responders compared to normal patients, while non-responder patients exhibited levels of this transcript that were only 1.5-fold higher than normal patients (FIG. 2B). Further analysis of this gene was pursued FKBP51 transcript levels showed a significant increase in isolated PBMCs treated with all of the FP concentrations tested (FIG. 3A). FP treatment induced an increase in FKBP51 protein levels in primary epithelial cells cultured from an EE patient esophageal biopsy. Following 24 hours of treatment with FP, western blot analysis indicated an approximately 3-fold increase in FKBP51 protein in these cells (FIG. 3B).

To further study the role of FKBP51 in epithelial cells, the TE-7 esophageal cell line was used to understand the expression patterns of FKBP51 following FP treatment. Induction of FKBP51 was observed in response to various doses of FP in TE-7 cells. FKBP51 protein levels showed a dose-dependent increase in response to FP treatment (FIG. 3C). The kinetics of FKBP51 Mrna and protein expression were examined following FP treatment. Transcript levels of FKBP51 were increased by 4 h post treatment and were further increased by 8 h, after which no further increase was observed (FIG. 3D, left). Similarly, FKBP51 protein levels in TE-7 cells increased in a time dependent manner, with maximum levels of protein observed by 24 h following treatment.

### Example 4

### Localization of FKBP51 in Patient Biopsy Samples

Patient biopsy samples were immunostained using antibody specific for FKBP51, and nuclei were stained with DAPI. In biopsies obtained from FP responder patients, the strongest signal was observed in the basal layer of the esophageal tissue. The signal was most strong in the nuclei of these cells, although some cytoplasmic staining was apparent. Weaker cytoplasmic staining was also observed in the cells near the luminal side of the biopsy. The localization pattern for FKBP51 was very similar in non-responder patients as well, with the strongest signal being within the basal layer of the esophageal biopsy. Staining with isotype control antibody confirmed that the observed signal was specific for the FKBP51 antibody.

The expression pattern of FKBP51 in patient biopsies was consistent with its being present in epithelial cells. To test whether the FKBP51 signal colocalized with epithelial cells, biopsy samples were stained with antibodies for both E-cadherin to mark epithelial cells and FKBP51. FKBP51 signal did in fact co-localize with E-cadherin signal, demonstrating that FKBP51 is expressed in the epithelial cells of patient biopsy samples.

### Example 5

### Impact of FKBP51 on Glucocorticoid- and IL-13 Signaling in Esophageal Epithelial Cells

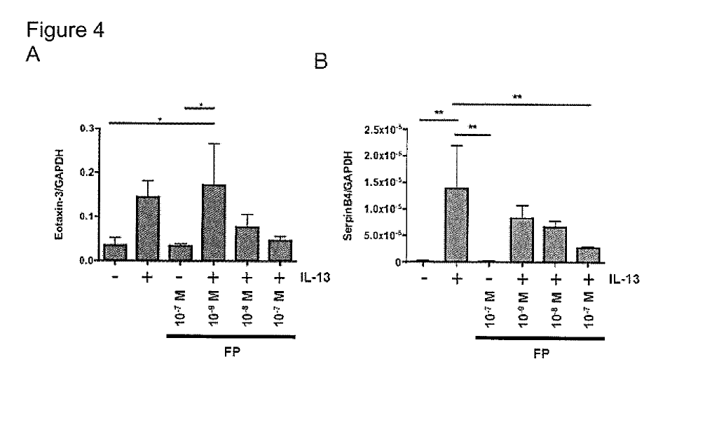

IL-13 signaling in esophageal epithelial cells is known to have a role in EE pathogenesis by inducing the EE transcriptome. Eotaxin-3, an eosinophil chemoattractant and activating factor, as well as serpinb4, a protease inhibitor, represent two target genes directly induced by IL-13 in esophageal epithelial cells. When TE-7 cells are treated with IL-13, an increase in eotaxin-3 and serpinb4 transcript levels is observed (FIG. 4A, B). When the cells are treated concomitantly with FP, the IL-13 mediated increase in transcript levels of these two genes is decreased in a dose-dependent manner (FIG. 4A, B).

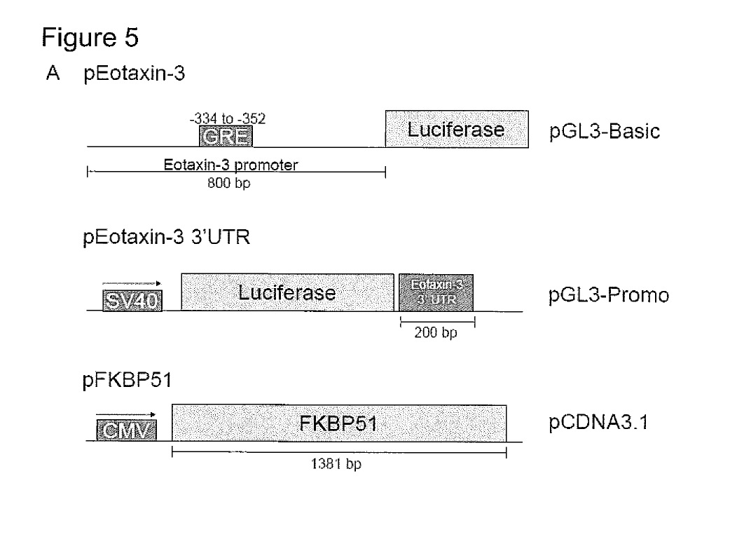

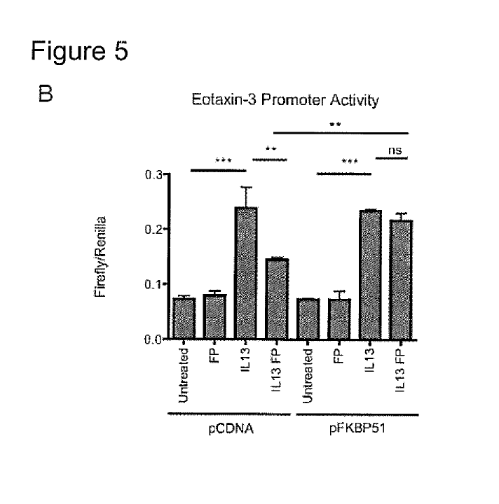

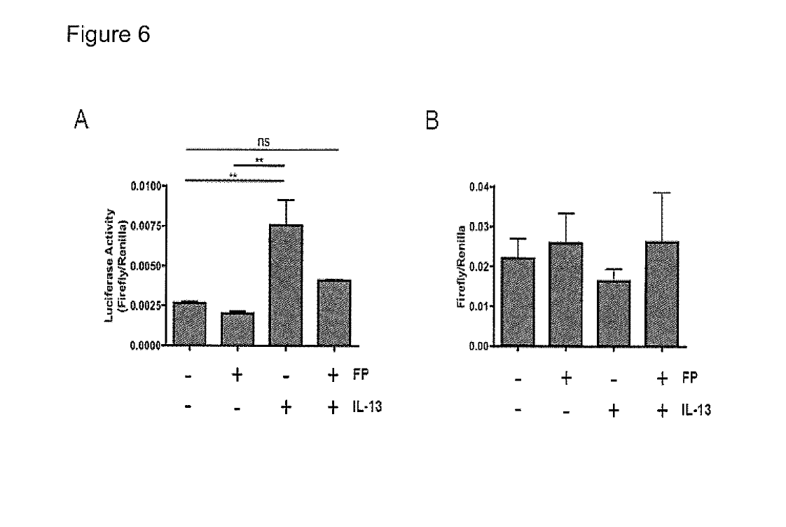

The contribution of the promoter and 3′ UTR sequences of eotaxin-3 to the control of its gene expression following IL-13 and FP treatment, was tested using TE-7 cells transiently transfected with a luciferase reporter construct. The cells were transfected with a luciferase reporter construct containing either 800 bp of the eotaxin-3 promoter 5′ of the firefly luciferase gene in the Pg13-Basic vector or a plasmid containing 200 bp of the eotaxin-3 3′ UTR downstream of the firefly luciferase gene in the Pg13-Promoter vector (FIG. 5A). The same cells were co-transfected with a plasmid containing Renilla luciferase under a constitutive promoter (Phrl-TK) that served as a control for transfection efficiency. Part of the observed decrease in IL-13 induced eotaxin-3 Mrna levels following FP treatment can be mediated through modulation of the promoter activity due to the presence of a putative glucocorticoid-responsive element (GRE) in this region (FIG. 5A). Experiments with the promoter sequence showed a decrease in IL-13-induced eotaxin-3 promoter activity upon concomitant FP treatment (FIG. 6A). Experiments with the 3′ UTR sequence did not show any modulation of Mrna stability upon IL-13 or FP treatment (FIG. 6B).

To test whether increased FKBP51 levels impacted the FP-mediated decrease in IL-13-induced eotaxin-3 promoter activity, TE-7 cells were transfected with either empty vector (Pcdna3.1) or an expression construction that contained FKBP51 under the control of the CMV promoter (Pfkbp51, FIG. 5A) to simulate high baseline levels of FKBP51. Cells transfected with the empty vector showed a 2-fold increase in eotaxin-3 promoter activity upon IL-13 treatment, and this was reduced by 50% when the cells were concomitantly treated with FP (FIG. 5B). However, when cells were transfected with the FKBP51 expression vector, the FP-mediated decrease in IL-13 induced eotaxin-3 promoter activity was abolished (FIG. 5B).

The various methods and techniques described above provide a number of ways to carry out the invention. Of course, it is to be understood that not necessarily all objectives or advantages described need be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several advantageous features, while others specifically exclude one, another, or several features, while still others specifically mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications is herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of determining the prognosis of eosinophilic esophagitis (EE) after glucocorticoid treatment in an individual, comprising:
determining the presence or absence of elevated FKBP51 expression; and
prognosing a responsive case of eosinophilic esophagitis after glucocorticoid treatment based upon the presence of elevated FKBP51 expression.

2. The method of claim 1, wherein where the presence of the glucocorticoid-responsive gene expression profile is indicative of a nonaggressive form of EE.

3. The method of claim 1, wherein where the presence of the glucocorticoid-responsive gene expression profile is indicative of a non-responsive form of EE.

4. A method for determining if an EE patient has been exposed to a steroid drug, comprising analysis of a gene expression profile of EE-associated genes, wherein the analysis comprises any or all of the genes in FIG. 1.

5. The method of claim 4, wherein the analysis comprises detection of presence or absence of elevated FKBP51 expression.

6. The method of claim 4, where the method identifies patient compliance with taking a medicine.

7. The method of claim 4, there the method determines the efficacy of a steroid drug.

8. A method of diagnosing an EE subtype comprising:
determining the presence or absence of at least one glucocorticoid-responsive transcript; and
diagnosing the EE subtype based upon the presence or absence of the transcript or transcripts.

9. The method of claim 8, wherein the EE subtype is responsive to FP treatment.

10. The method of claim 8, wherein the glucocorticoid-responsive transcript includes the expression of a gene described in FIG. 1(B) herein.

11. The method of claim 8, wherein the glucocorticoid is fluticasone propionate.

12. (canceled)

13. (canceled)

14. (canceled)

15. (canceled)

16. (canceled)

17. (canceled)

18. (canceled)

19. (canceled)

20. (canceled)
